(19) United States
(12) Patent Application Publication
(10) Pub. No.: US 2011/0174992 A1
(43) Pub. Date: Jul. 21, 2011

(54) Irradiation system for door handle

(76) Inventor: Masami Sakita, San Francisco, CA (US)

(21) Appl. No.: 12/807,521

(22) Filed: Sep. 8, 2010

Related U.S. Application Data
(63) Continuation-in-part of application No. 12/657,384, filed on Jan. 19, 2010, now abandoned.

(51) Int. Cl. A61L 2/10

(52) U.S. Cl. 250/492.1

(57) ABSTRACT

The door handle irradiation system includes at least one local subsystem that includes at least one door handle irradiation hardware, at least one motion detectors, at least one door open/close state sensor; and at least one device to program the local subsystem. The device to program the local subsystem is connected to the local subsystem by a wired or wireless communication means. The local subsystem includes a control means, door handle irradiation hardware, at least one motion detector and at least one door open/closed state sensor. The door handle irradiation hardware includes a housing that contains a plurality of germicidal lamps. The housing is generally a letter C- or reverse letter C-shaped, and comprises a vertical part and two horizontal parts, contiguous to one another, and each part contains at least one germicidal lamp that emits ultraviolet rays.

### RELATED APPLICATION

This application is a continuation-in-part of application Ser. No. 12/657,384 filed on Jan. 19, 2010 entitled “Irradiation System for Door and Faucet Handles.”

### FIELD OF THE INVENTION

This invention relates generally to a device and a system used to irradiate the door handle.

### BACKGROUND OF THE INVENTION

It is reported that antibiotic-resistant bacterium called NDM-1 has become increasingly common in India and Pakistan, and is being found in patients in Britain and the United States in the patients who have been treated in these countries. Antibiotic-resistant bacteria have been causing a big problem to the hospitals in the United States even before the finding of the new strain of bacteria. It has been reported in the news that 100,000 people die every year in the hospital from hospital-acquired infections caused by antibiotic-resistant bacteria in the United States and that it is costing billions of dollars. In order to combat this problem, hospital administrators in some hospitals have started to enforce a strict hand-washing regimen in conjunction with peer pressures with some carrots and sticks to hospital workers. According to the news, the number of cases of the infectious diseases decreased somewhere between 30 to 70 percent in these hospitals that enforces the strict hand-washing regimen. Though hand-washing is very important especially in the hospital and should be encouraged to every worker, hand washing alone will not fully solve the problem, and thus other measures will also have to be taken to alleviate the problem.

### OBJECTS OF THE INVENTION

An object of this invention is the provision of a device that irradiates the door handle without harming those who use the door handle.

In order to achieve the above mentioned object, the proposed door handle irradiation system utilizes germicidal ultraviolet lamps. According to Kowalski et at in “Mathematical Modeling of Ultraviolet Germicidal Irradiation for Air Disinfection,” in Quantitative Microbiology (Springer) 2 (3): 249-270, at wavelength of 2,437 Angstroms (254 nm) ultraviolet ray will brake the molecular bonds within micro-organismal DNA, producing thymine dimmers in their DNA thereby destroying them, and rendering them harmless or prohibiting growth and reproduction. The idea involved in this invention is to develop a system that is able to irradiate door handles that are frequently touched by hospital workers, patients and visitors. The same system that is proposed in this invention may also be used in other facilities such as restaurant bathrooms.

### SUMMARY OF THE INVENTION

The preferred embodiment of a door handle irradiation system includes at least one local subsystem that includes at least one door handle irradiation hardware, at least one motion detectors, at least one door open/close state sensor; and at least one “means to program the local subsystem”. The “means to program the local subsystem” is connected to the local subsystem by a wired or wireless communication means. The local subsystem includes a control means, door handle irradiation hardware, at least one motion detector and at least one door open/closed state sensor.

The door handle irradiation hardware includes a housing that contains a plurality of germicidal ultraviolet lamps, ballasts, and starters and necessary wiring for powering the local subsystem. The housing is generally a letter C- or reverse C-shaped, comprising an upper horizontal part, a vertical part and a lower horizontal part, and each part contains at least one germicidal lamp that emits ultraviolet rays. The letter C- or reverse C-shaped housing is affixed to the door in such a manner that the housing will enclose the door handle when viewed from the front, and the ultraviolet rays emitted by the germicidal lamps will light the door handle.

The control means of the local subsystem includes an integrated circuit chip computer; sensors that trigger switching on/off activity of the germicidal lamps; and control and communication software included in the ROM (or EPROM) of the computer.

The software of the “means to program the local subsystem” defines how the local subsystem is operated at a time by a day-of-the-week based time-of-day scheme that is defined by a table, in which the entries to the table are the start time of an operational method, and operational parameters to the specified operational method. The operational methods include the detector-activated method and the pre-timed method with detector-activated protection. The “means to program the local subsystem” is able to set and change the operational method and the operational parameters of the local subsystem.

### BRIEF DESCRIPTION OF THE DRAWINGS

The above description and other objects and advantages of this invention will become more clearly understood from the following description when considered with the accompanying drawings. It should be understood that the drawings are for purposes of illustration only and not by way of limitation of the invention. In the drawings, like reference characters refer to the same parts in the several views:

FIG. 1 is a schematic diagram of the preferred embodiment of the door handle irradiation system;

FIG. 2A is a front view, FIG. 2B a side view, and FIG. 2C a bottom view of the local subsystem hardware of the preferred embodiment of the door handle irradiation system and a door handle that is irradiated;

FIG. 3 is a cross-sectional view taken along A-A of FIG. 2A;

FIG. 4 is a cross-sectional view taken along B-B of FIG. 2A;

FIG. 5 is a schematic diagram showing the control means of the local subsystem of the preferred embodiment;

FIG. 6 is a schematic time-space diagram showing key time-space points involved in switching on the germicidal lamp in the detector-triggered method;

FIG. 7 is a schematic diagram showing the means to program a local subsystem of the preferred embodiment; and

FIG. 8A is a front view, FIG. 8B a side view, and FIG. 8C a bottom view of the local subsystem hardware of an alternative embodiment and a door handle.

### DETAILED DESCRIPTION OF THE INVENTION

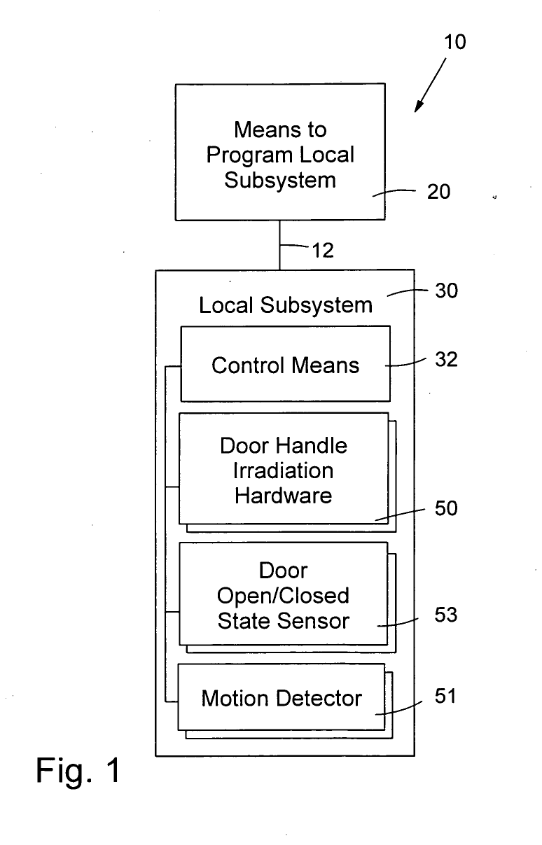

As shown in FIG. 1, the preferred embodiment of a door handle irradiation system 20 includes at least one local subsystem 30 and at least one “means to program the local subsystem” 20 wherein the local subsystem is connected to the means to program local subsystem by a wired or wireless communication means 12. The local subsystem 30 includes a control means 32, at least one door handle irradiation hardware 50, at least one door open/close state sensor 53, and at least one motion detector 51.

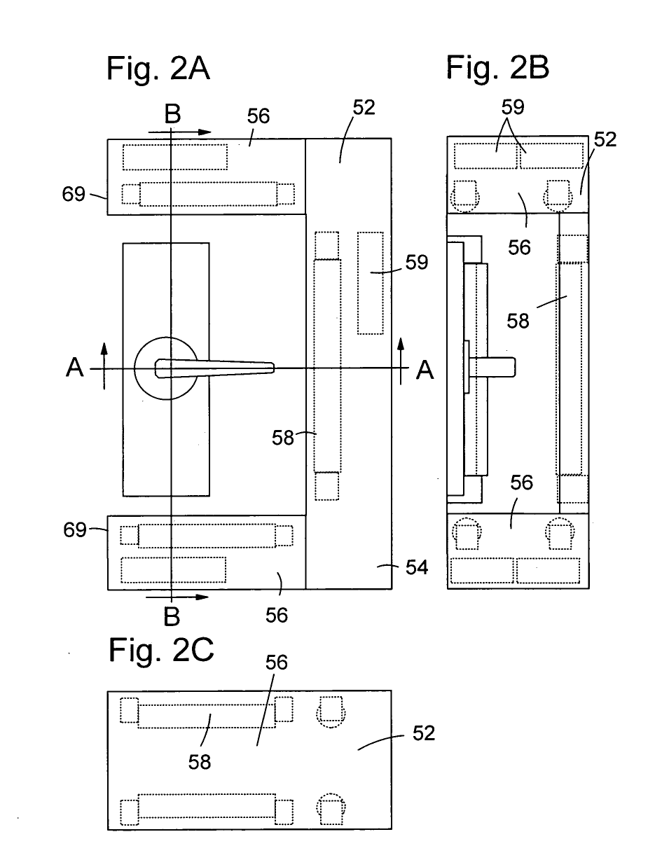

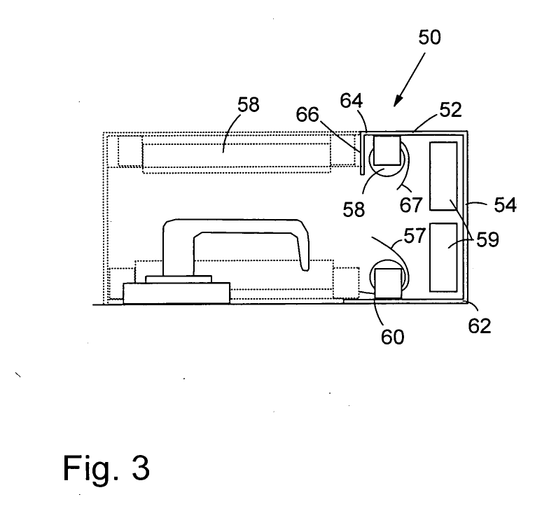

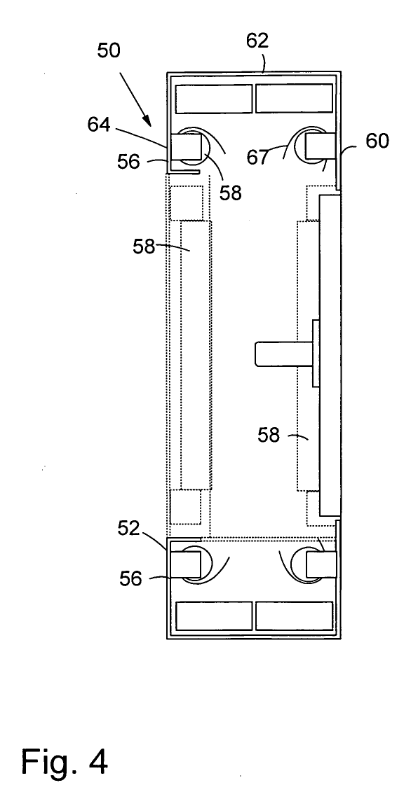

As shown in FIGS. 2A through 4, the door handle irradiation hardware includes a housing 52 that includes a plurality of germicidal lamps 58, ballasts 59, starters and necessary wiring for powering the local subsystem. The housing 52 is generally a letter C- or reverse letter C-shaped when looked from the front as shown in FIG. 2A. The housing 52 comprises two horizontal parts 56 and a vertical part 54, and each part contains at least one germicidal lamp 58 that emits ultraviolet rays. The housing 52 is affixed to the door in such a manner that the housing will enclose the door handle when viewed from the front, and the ultraviolet rays emitted by the germicidal lamps will light the door handle.

Each part of the housing 52 comprises a base 60, a rear wall 62, a top 64 and a front eave 66, and each germicidal lamp 58 has a reflector 67 to project the ultraviolet ray to the direction of the door handle. In addition, each of the two horizontal parts includes a side wall 69. One germicidal lamp 58 is disposed in the space created by the rear wall 62, the top 64 and the front edge 66 in each part of the housing 52. Another germicidal lamp 58 is disposed at the base 60 by the rear wall 64 in each part of the housing 52.

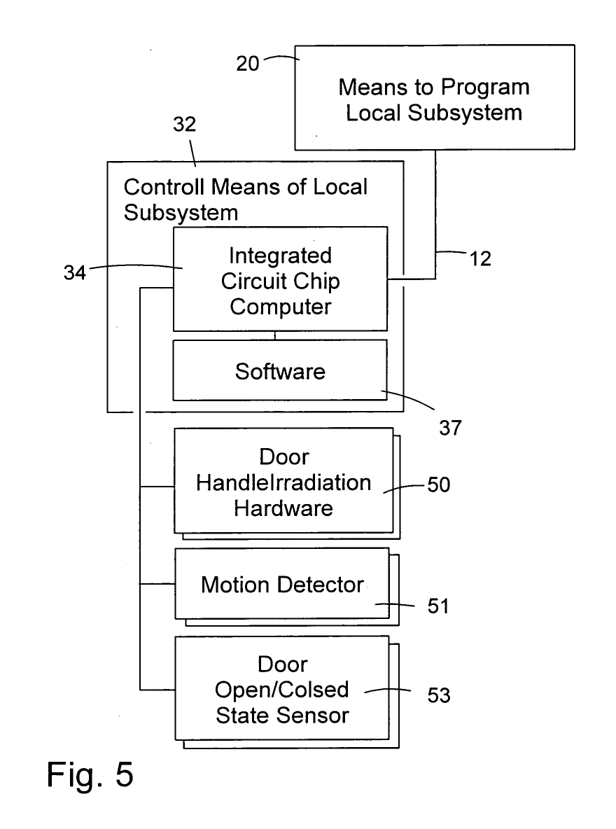

As shown in FIG. 5, the control means 32 of the local subsystem 30 includes an integrated circuit chip computer 34 that includes a CPU, RAM, ROM and input/output interface; and software 37 included in the ROM (or EPROM) of the computer. The software 37 of the means to program stored in the circuit chip computer defines the “day-of-the-week based time-of-day table”, in which the entries to the table are the start time of an operational method, and operational parameters to the specified operational method. The operational methods include the detector-triggered switching method and the “pre-timed switching method with detector-trigger protection”. The “means to program the local subsystem” is able to set and change the operational method and the operational parameters of the local subsystem. Each of the control methods is able to switch on/off all or a part of the germicidal lamps. The control means 32 of the local subsystem 30 may be installed within the housing 52 of the local subsystem hardware 50, or any other place that would be proper for installing such a means.

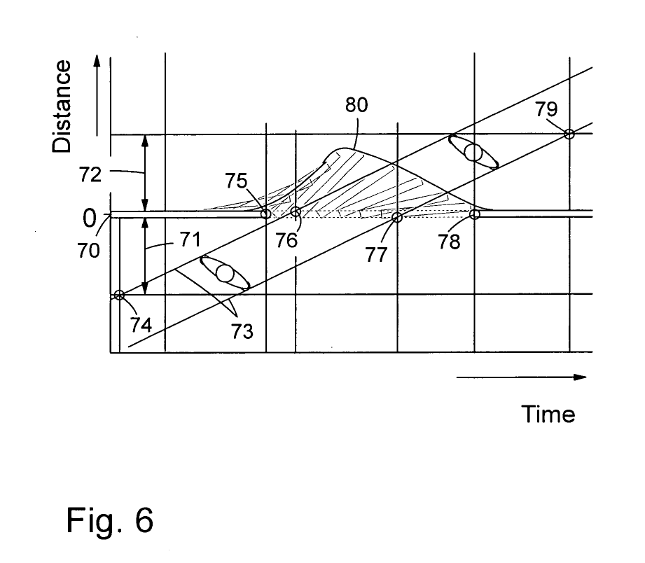

In FIG. 6 that shows the time-space diagram involved when a person gets into a room from the hallway (side A of the door) using the door that is equipped with the door handle irradiation system. As the figure shows, the door opens to the inside of the room (side B of the door) as most doors do. The time-space diagram shows key events involved in switching on the germicidal lamps in either side of the door, wherein the x-axis represents time and the y-axis represents space wherein “0” indicates the location where the door exists. The range covered by the motion detector is indicated by 71 on side A that represents the hallway side of the door, and 72 on side B that represents the room side of the door. The outer envelopes of the trajectory of the person who uses the door are represented by lines 73, and the outer envelope of the trajectory of the tip of the horizontal end of the door is represented by 80. FIG. 6 also shows the point 74 that indicates the motion detection starting in side A; the point 75 that indicates the door open/closed state sensor changing the state from the door closed to door open; the point 76 that indicates the motion detection start time in the side B; the point 77 that indicates the motion detection end time in side A; the point 78 that indicates the door close time; and the point 79 that indicates motion detection end time in side B.

In the detector-triggered switching method, which is used during the busy period during which period, the door is opened and closed frequently, a predefined group of the germicidal lamps of side A of the door are switched on for a predetermined time period after a series of events such that (1a) the motion detector 51 in side A of the door changed from the non-detection state to the detection state; (2a) the door open/close state sensor 53 changed from the closed state to the open state; (3a) the motion detector 51 in side A of the door changed from the detection state to the non-detection state; and (4a) the door open/close state sensor 53 changed from the open state to the closed state, are observed. In the same door open/close activity, a predefined group of the germicidal lamps of side B of the door are switched on for a predetermined time period after a series of events such that (1b) the door open/close state sensor 53 changed from the closed state to the open state; (2b) the motion detector 51 in side B of the door changed from the non-detection state to the detection state; (3b) the door open/close state sensor 53 changed from the open state to the closed state and the motion detector; and (4b) the motion detector 51 in side B of the door changed from the detection state to the non-detection state, are observed.

The sequence of time events that take place while a person walk out of the room through the door from side B to side A will be the same as that observed as that observed while a person walks into the room through the door from side A to side B, albeit the events that take place in side A will take place in side B, and vice versa. It implies that the side to which the door opens does not make difference in the time sequence of the events, and that if the sequence (1a) through (1d) above is observed, the germicidal lamps may be lit in that side of the door handle irradiation hardware 50, and the sequence (1b) through (4b) above is observed, the germicidal lamps may be lit in that side of the door handle irradiation hardware 50.

In the pre-timed switching method with detector-trigger protection, which is used during the non-busy period, the germicidal lamp 58 of one side of the door is switched on for a predetermined time period at a given time interval if the door open/closed state sensor 53 is at the closed state and the motion detector 51 of that side is activated.

In either of the operation methods, the germicidal lamps 58 will be switched off as soon as the motion detector 51 on the same side of the door on which the germicidal lamps 58 are installed is activated. The already-lit germicidal lamps 58 on either side of the door will be switched off as soon as the door open/closed state sensor 53 changes from the door closed state to the door open state. The germicidal lamps 58 on either side of the door will not be lit if the door open/closed state sensor 53 indicates that the door is open, or the motion detector 51 is activated; i.e., detecting a human on either side of the door. In the operation of the local subsystem, the control means 32 is able to switch on/off the germicidal lamp 58 for each lamp independently of the other. For example, the germicidal lamp 58 affixed closer to the door surface in the vertical part of the housing may not be lit at all during the busy period so that nobody will see the ultraviolet rays even from far away.

The door open/close state sensor 53 uses an electric circuit that opens when the door is in the open state, and closes when the door is in the closed state. The door open/close state sensor 53 determines that the door handle is being used when the circuit is in the open state. The motion detector 51 that detects presence of a person in front of the door emits and receives a microwave, and by measuring the time it takes in reflection determines the presence of a person.

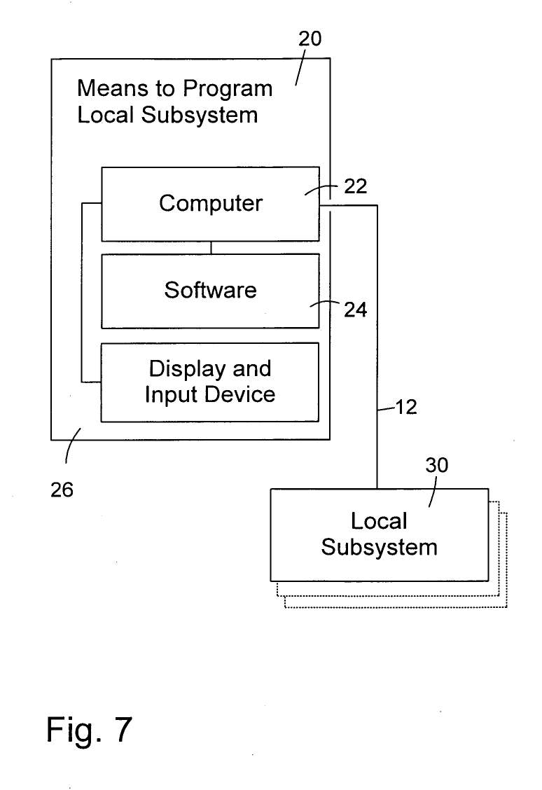

As shown in FIG. 7, the means 20 to program local subsystem includes a computer 22, software 24, a communication means for connection with the local subsystem, and a display and input device 26. By using the means 20 to program local subsystem, one is able to monitor all activities of motion detector, the door open/close state sensor, and the germicidal lamp lamps, etc., of the local subsystem on a real time basis, and record all these events, in addition to program all parameters of a local subsystem for each door handle. In an alternative design, the means 20 to program the local subsystem 30 is the software that is included in the computer 22 such as a smart phone instead of the whole unit that includes the hardware and the software.

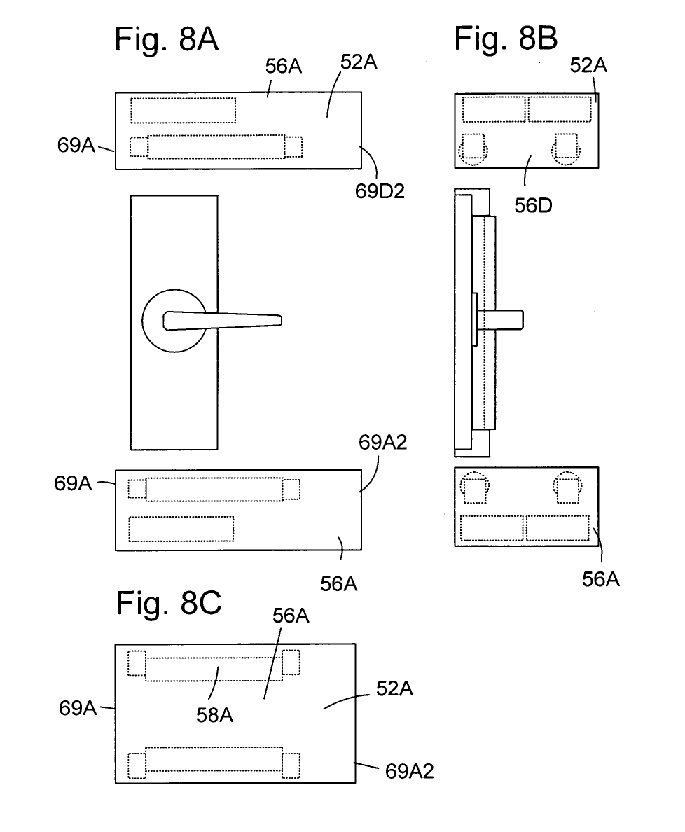

As shown in FIGS. 8A through 8C, this alternative embodiment includes local subsystem hardware 52A that includes a housing that has two horizontal parts 56A, one of which is attached to the door above the door handle facing the door handle, and the other is attached to the door below the door handle facing the door handle. Each of the two parts contains at least one germicidal lamp 58A, ballasts, starting light and wirings for power system. The housing 52A of the local subsystem of this alternative embodiment is generally the same as that of the preferred embodiment except that the alternative embodiment does not have the vertical part and that the horizontal part has an extra side wall 69A2 in addition to the side wall 69A.

Another alternative embodiment is a stand alone local subsystem without the means to program local subsystem. In such a system, the control parameters of the local subsystem are all predetermined, and cannot be changed by the user. Otherwise, this alternative embodiment is generally identical to the local subsystem of the preferred embodiment.

The invention having been described in detail in accordance with the requirements of the U.S. Patent Statutes, various other changes and modifications will suggest themselves to those skilled in this art. It is intended that such changes and modifications shall fall within the spirit and scope of the invention defined in the appended claims.

I claim:

1. A door handle irradiation system including at least one local subsystem and at least one means to program said local subsystem wherein
said local subsystem includes a control means, at least one door handle irradiation hardware, at least one motion detector, and at least one door open/closed state sensor,
said means to program said local subsystem and said local subsystem is connected by a communication means,
said door handle irradiation hardware includes a housing,
said housing contains a plurality of germicidal lamps, and
said germicidal lamp emits ultraviolet rays.

2. A door handle irradiation system as defined in claim 1 wherein
said housing includes first and second horizontal parts,
each horizontal part of said housing includes at least one of said germicidal lamp, and
said germicidal lamps in said first and second horizontal parts face a door handle that is irradiated by said germicidal lamps.

3. A door handle irradiation system as defined in claim 2 wherein
said housing includes a vertical part in addition to said first and second horizontal parts wherein said vertical part is connected to said first horizontal part in one end and connected to said second horizontal part in the other end to form a letter C- or reverse C-shaped housing.

4. A door handle irradiation system as defined in claim 1 wherein
said control means of said local subsystem include software,
said software includes a day-of-the-week based time-of-day table that defines starting times of operational methods and their parameters wherein said operational methods include a sensor triggered switching method and sensor-protected pre-timed switching method.

5. A door handle irradiation system as defined in claim 1 wherein
said means to program local subsystem includes a computer, and a display and input device,
said computer includes software used to monitor and control said local subsystem,
said software used to monitor and control local subsystems includes operational methods and operational parameters, and
said means to program said local subsystem is able to set or change said operational method and said operational parameters.

6. A door handle irradiation system including at least one local subsystem and software used to program said local subsystem wherein
said local subsystem includes a control means, at least one door handle irradiation hardware, at least one motion detector, and at least one door open/closed state sensor,
said means to program said local subsystem and said local subsystem is connected by a communication means,
said door handle irradiation hardware includes a housing,
said housing contains a plurality of germicidal lamps, and
said germicidal lamp emits ultraviolet rays.

7. A door handle irradiation system as defined in claim 6 wherein
said housing includes first and second horizontal parts,
each horizontal part of said housing includes at least one of said germicidal lamp, and
said germicidal lamps in said first and second horizontal parts face a door handle that is irradiated by said germicidal lamps.

8. A door handle irradiation system as defined in claim 6 wherein
said housing includes a vertical part in addition to said first and second horizontal parts wherein said vertical part is connected to said first horizontal part in one end and connected to said second horizontal part in the other end to form a letter C- or reverse C-shaped housing.

9. A door handle irradiation system as defined in claim 6 wherein
said control means of said local subsystem include software,
said software includes a day-of-the-week based time-of-day table that defines starting times of operational methods and their parameters wherein said operational methods include a sensor triggered switching method and sensor-protected pre-timed switching method.

10. A door handle irradiation system as defined in claim 6 wherein
said software used to program said local subsystem is able to set or change said operational method and said operational parameters.

11. A door handle irradiation system including control means and at least one door handle irradiation hardware wherein
said door handle irradiation hardware is connected to at least one motion detector and at least one door open/closed state sensor;
said door handle irradiation hardware includes a housing,
said housing includes at least two horizontal parts,
said housing contains a plurality of germicidal lamps,
said germicidal lamp emits ultraviolet rays.

12. A door handle irradiation system as defined in claim 11 wherein
said housing includes a vertical part in addition to said first and second horizontal parts wherein said vertical part is connected to said first horizontal part in one end and connected to said second horizontal part in the other end to form a letter C- or reverse C-shaped housing.

13. A door handle irradiation system as defined in claim 11 wherein
said control means of said local subsystem include software,
said software includes a day-of-the-week based time-of-day table that defines starting times of operational methods and their parameters wherein said operational methods include a sensor triggered switching method and sensor-protected pre-timed switching method.
